(19) United States
(12) Patent Application Publication
(10) Pub. No.: US 2016/0325038 A1
(43) Pub. Date: Nov. 10, 2016

(54) COVERING DEVICE AND APPARATUS FOR MONITORING THE CONNECTORS OF A HOSE SYSTEM

(71) Applicant: FRESENIUS MEDICAL CARE DEUTSCHLAND GMBH, Bad Homburg (DE)

(72) Inventor: Alexander SCHROERS, Frankfurt (DE)

(73) Assignee: Fresenius Medical Care Deutschland GmbH, Bad Homburg (DE)

(21) Appl. No.: 15/109,892

(22) PCT Filed: Feb. 13, 2015
(86) PCT No.: PCT/EP2015/053146

§ 371 Date: Jul. 6, 2016

(30) Foreign Application Priority Data
Feb. 14, 2014 (DE) 10 2014 001 898.9

(51) Int. Cl. A61M 1/36; G01M 3/16; A61B 5/02

(52) U.S. Cl. CPC A61M 1/3656; A61B 5/02042; A61M 1/367; G01M 3/16; A61M 2205/15; A61M 2209/088; A61B 2562/029

(57) ABSTRACT

A covering mechanism for covering a hose system that can be connected to a patient with a sheet having a liquid sensor is described. The liquid sensor is disposed inside a fold in the sheet, so that any leakage at the connectors of the hose system can be detected by this liquid sensor, even when the hose system is covered by the covering mechanism.

The invention relates to a covering mechanism of a hose system, which can be attached to a patient and with which leakage can be detected.

Moisture sensors in the form of patches for monitoring a patient's vascular access are known from WO 2006/008866 A1 and U.S. Pat. 6,445,304 B1, for example. A moisture sensor in the form of a cuff that can be wrapped around the hose system is also known in the prior art.

In the field of medical technology, there are various known devices with which liquids can be taken from a patient via a vascular access, e.g., a catheter, and via a hose system and can also be returned to the patient again via the same routes.

One example is extracorporeal blood treatment machines, for example, dialysis machines or cell separators, which necessitate access to the patient's vascular system to create an extracorporeal blood circulation. In such treatments, it is customary for the vascular access and the neighboring hose system to be wrapped in a protective sheathing, hereinafter referred to as a textile field. This should preserve the most sterile possible environment for the vascular access and the hose system. This textile field is supplied, together with the blood hose system, in part by manufacturers of blood hose systems in the form of a cloth or as a field of gauze. It is used by the nursing staff as a substrate and sheathing for the hose system.

If the vascular access and the hose system are covered by the sterile field, this may result in leakage in the vascular access or in the Luer-Lock connectors of the hose system not being detectable by the nursing staff at an early point in time. Such leakage can occur, for example, due to slippage of the cannula out of the patient's blood vessel or when the connection of the Luer-Lock connectors is loosened. Especially in extracorporeal blood treatments, even minor leakage can lead to unwanted blood losses because of the high flow rates. The cover must therefore be removed and the access must be inspected at fixed intervals of time.

Various devices of different designs are known in the prior art for monitoring the vascular access. The known monitoring devices usually rely on the safety systems that are present as the standard in blood treatment machines and trigger an immediate interruption in the extracorporeal blood circulation whenever the vascular access is not in order.

Such devices for monitoring may include, for example, devices for detection of moisture or devices with optical sensors, which detect the leakage of blood out of the puncture site or connectors of the hose system. The moisture sensors are designed as a pad, for example, which is placed on the patient's skin, or as a cuff which is wrapped around the connectors of the hose system.

These known devices are designed only for small-area application directly at the puncture site and cannot fulfill a covering and protecting function. Furthermore, these known devices may lead to false alarms when smaller amounts of liquid escape from the end of the hose when blood hoses are connected or when the connectors are being manipulated by the nursing staff.

The object of the present invention is to make available a device for covering a hose system, which will permit early detection of a leak, where this device should be independent of the embodiment of the hose system. False alarms should be avoided in the case of leaks that may occur in operations involving the hose system.

According to the teaching of the present invention, this object is achieved by a covering mechanism according to claim 1 and a device for monitoring a patient's access according to claim 15. Advantageous embodiments are the subject matter of the dependent claims.

The covering mechanism according to the invention has a sheet and a fluid sensor for detecting a leak at the vascular access or on the hose system during a blood treatment.

The sheet also has a kinked edge. A gusseted pocket is formed when the sheet is folded along the kinked edge. The gusseted pocket is located in the area of the covering mechanism, in which two layers of the sheet are situated one above the other. The gusseted pocket has a top and a bottom partial area, such that the bottom partial area is the partial area closer to the patient.

In general, the bottom arrangement should always denote the orientation closer to the patient than the top arrangement.

The liquid sensor is disposed on the sheet in such a way that it is disposed along the kinked edge in the resulting gusseted pocket after the folding of the sheet. At least a part of the liquid sensor may be disposed on the bottom partial area of the gusseted pocket.

One advantage of the covering mechanism according to the invention is that it facilitates patient care during an extracorporeal blood treatment and increases the reliability of the treatment.

In another embodiment, the sheet has a second kinked edge, which runs largely parallel to the first kinked edge and is formed in the opposite direction from the first kinked edge. When the sheet is folded along the two kinked edges, the result is a zigzag fold with a first gusseted pocket and a second gusseted pocket, which open in opposite directions,

Due to the zigzag folding, the folding and unfolding of the gusseted pockets may be accomplished by lateral pulling and/or pushing on the sheet.

In another embodiment, the sheet has at least four kinked edges, with the neighboring kinked edges always being formed in opposite directions and a zigzag fold with at least three gusseted pockets being the result of the folding.

The liquid sensor is preferably situated in the bottom gusseted pocket.

In one embodiment, the liquid sensor sits on at most one kinked edge of the sheet. This minimizes the risk of damage to the liquid sensor due to kinking.

In one embodiment, the partial areas of the gusseted pockets are wider than the hose system to be covered. If the covering mechanism has more than one gusseted pocket, then the width of the partial area of the gusseted pocket is defined by the distance between two kinked edges. The width of the partial area of the gusseted pocket may be between 5 and 15 cm, preferably between 8 and 12 cm.

In one embodiment, the sheet may be a textile fabric, an absorbent film and/or a nonwoven, which offers a pleasant feel on the skin.

In another embodiment, the sheet may be impermeable to moisture on one side, which thus prevents penetration of liquid through an absorbent fabric and thus also prevents the false alarms from being triggered.

In one alternative embodiment, the sheet may be impermeable to moisture and may be coated paper, for example, so that the device according to the invention can be produced easily and reliably.

The sheet may be sterilizable.

The sheet may be sterile on at least one side.

The sheet may be sterilized.

In one embodiment, the sheet may be between 400 and 600 mm long and between 350 and 550 mm wide.

In one embodiment the liquid sensor may be between 100 and 200 mm long and between 100 and 200 mm wide.

The sheet may have markings which indicate to the user how the sheet is to be disposed with respect to the vascular access. These markings may indicate to the user which position is to be disposed as close as possible to the location of the vascular access. In addition, markings may indicate to the user where and in which direction the sheet is to be folded and unfolded in pulling it apart and folding it together.

These markings may be made in the form of arrows.

In one embodiment of the covering mechanism, the liquid sensor and the sheet are fixedly joined to one another. The liquid sensor may be integrated into the sheet. In one alternative embodiment, the liquid sensor may be attached to the sheet. The liquid sensor may be sewn or glued to the sheet. The position of the liquid sensor should be centrally on the edge of the sheet. Thus the sheet together with the liquid sensor can be advanced as close as possible to the outlet opening of the catheter. A cutout in the sheet, at the end of which the liquid sensor is located, is also possible so that the sheet surrounds the vascular access from all sides.

The liquid sensor may have an electrically conductive structure. The electrically conductive structure may have one or more conductors.

The electrically conductive structure may be printed on a woven or nonwoven fabric.

The electrically conductive structure may be woven into the woven fabric.

For electrical contacting of the liquid sensor, the covering mechanism according to the invention has electrical connecting contacts. The number of connecting contacts depends on the design of the liquid sensor. Alternatively, electrical connecting lines may also lead out of the liquid sensor and can be connected to a device for monitoring a patient's vascular access.

In one particular embodiment of the covering mechanism according to the invention, a barrier of an absorbent material without a measurement function is applied to the edge of the liquid sensor to prevent lateral penetration of liquid into the fold.

The barrier should not have any contact with the liquid sensor. The distance between the liquid sensors may be between 0.5 and 1 cm, for example.

The barrier may be situated inside the gusseted pocket, i.e., in the folded state of the sheet, the barrier is covered by the top partial area of the gusseted pocket or the barrier is disposed outside of the gusseted pocket and connected to the kinked edge of the gusseted pocket.

The device according to the invention for monitoring the connectors of a hose system, in particular for monitoring the connectors of the blood hose system in an extracorporeal blood treatment has the covering mechanism according to the invention for detection of moisture, namely the sheet having the liquid sensor which is connected to the monitoring device. The monitoring device may trigger an acoustic and/or optical and/or tactile alarm when moisture is detected. A control signal may also be generated for intervention into the control of the device, which supplies a fluid to the patient through a hose line and/or removes a fluid from the patient.

Additional embodiments and advantages of the invention are explained in greater detail below on the basis of an exemplary embodiment and a few figures.

They show

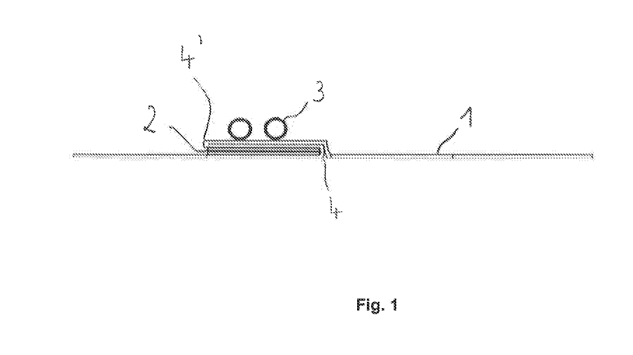

FIG. 1: a cross section through a sheet and the liquid sensor is protected by the folding of the sheet, the blood hose system sits on the sheet above the liquid sensor.

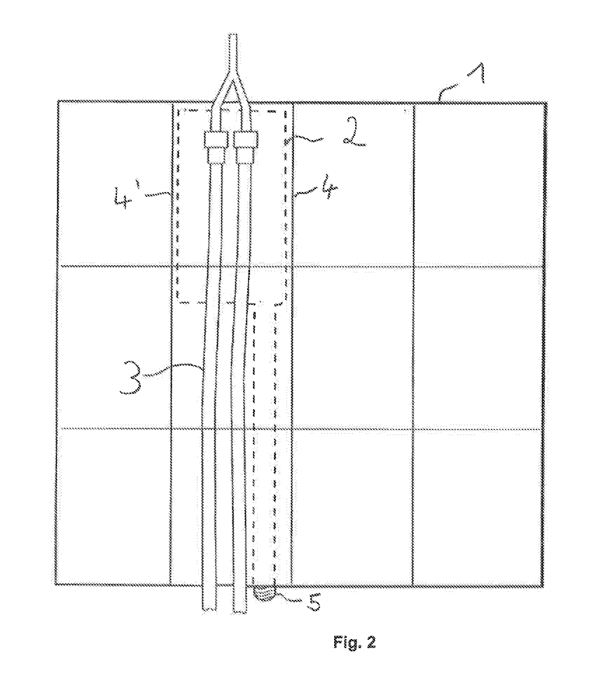

FIG. 2: a top view of the arrangement in FIG. 1, the blood hose system being disposed on the fold. The liquid sensor is in the fold (dotted line).

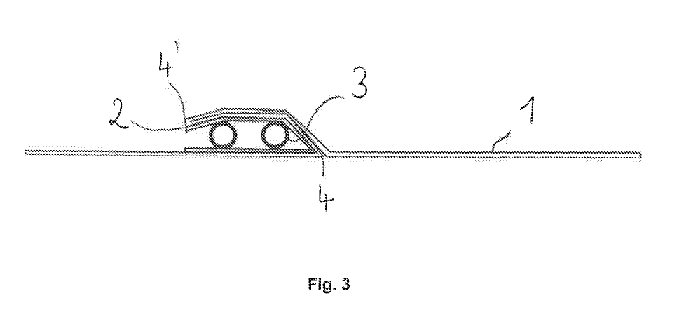

FIG. 3: a cross section through a sheet as shown in FIG. 1, the blood hose system disposed on the liquid sensor, covered by a fold.

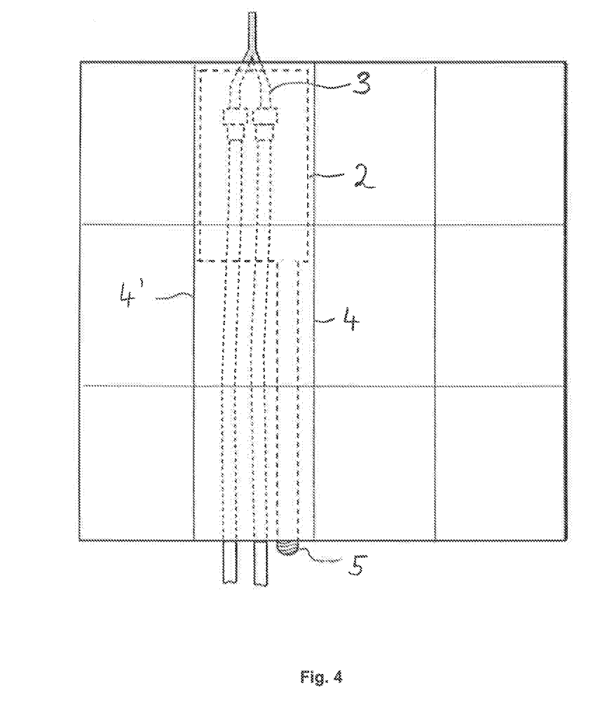

FIG. 4: a view of the arrangement in FIG. 3, with the blood hose system (dotted line) covered by a fold.

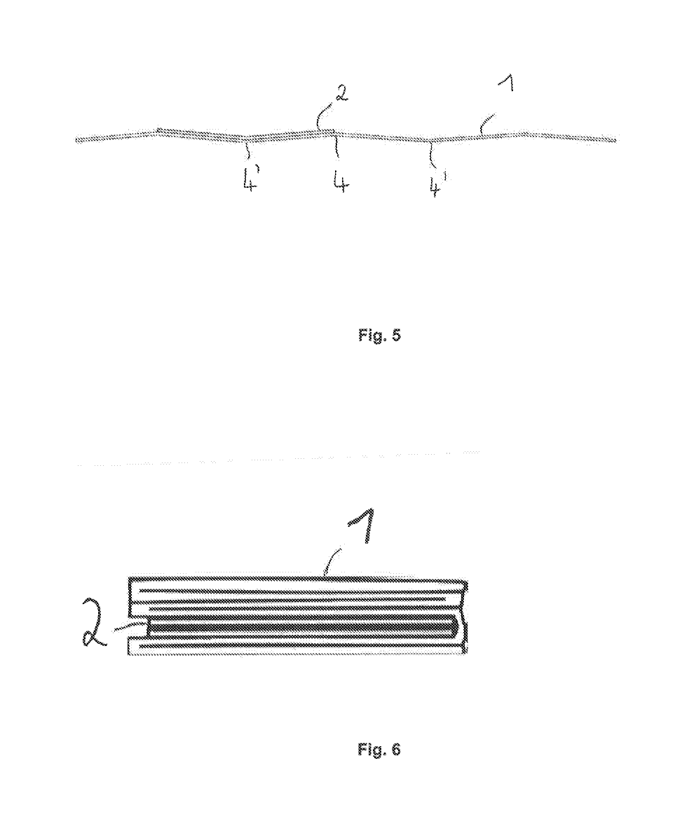

FIG. 5: a cross section through a sheet with a liquid sensor, unfolded, 5 folds 4-4′ with bending directions of the fold indicated.

FIG. 6: a cross section through the sheet with 5 kinked edges.

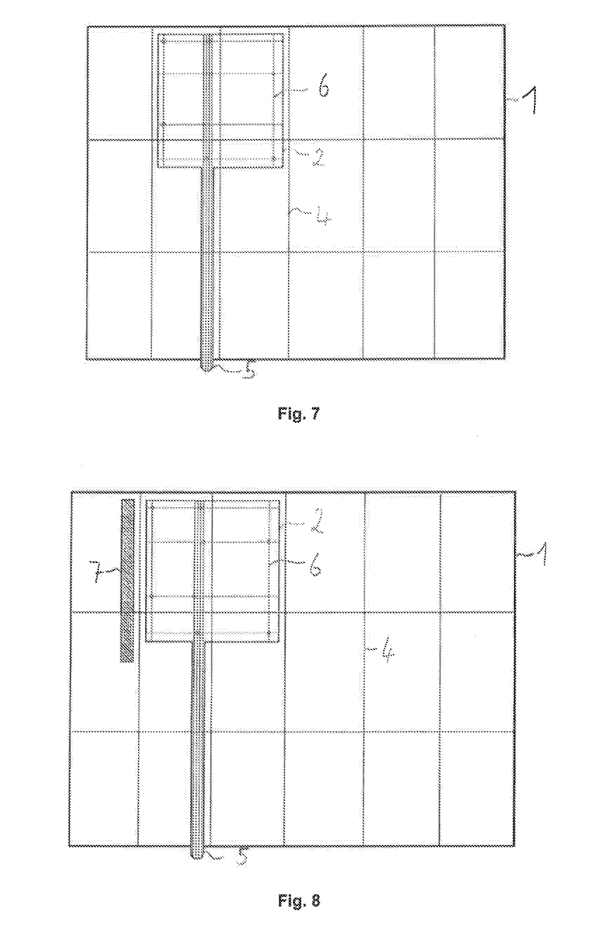

FIG. 7: a top view of the sheet with 5 folds with a liquid sensor unfolded with printed conductors and connecting contact.

FIG. 8: a top view of a sheet with a liquid sensor, unfolded with a lateral barrier.

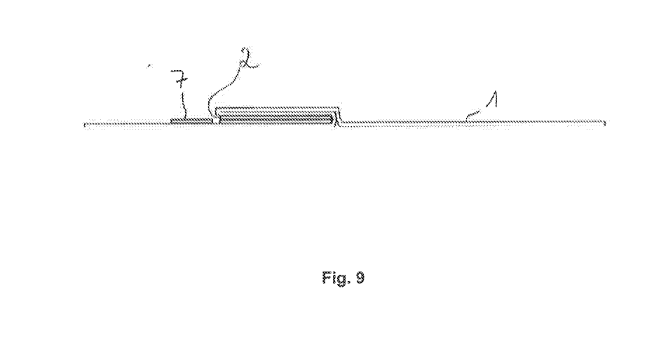

FIG. 9: a cross section through a sheet with a liquid sensor and with a lateral barrier, folded up.

FIG. 1 shows a covering mechanism according to the invention having a sheet 1 which is coated paper in this exemplary embodiment, preferably sterilizable, flexible and water-repellent. A first kinked edge 4 is created in the coated paper, permitting folding of the paper 1 upward, so that a folded part of the coated paper as shown in FIG. 1 comes to lie in parallel on the remaining part of the coated paper after the kinked edge 4′ and thus forms a gusseted pocket. In this context, “upward” is intended with respect to the arrangement of the covering mechanism in FIG. 1. A liquid sensor 2 is pressed against the coated paper 1 in this exemplary embodiment and is cut approximately at the center by the kinked edge 4. One-half of the sensor 2 is therefore folded onto the other half of the sensor 2 when the coated paper is folded over. The coated paper additionally has a second kinked edge 4′ on which the coated paper can be folded over in the opposite direction. After this second folding, a second gusseted pocket is formed, a type of cover consisting of a double layer of coated paper completely covering the sensor 2. In the arrangements shown in FIGS. 1 and 2, the covering mechanism lies on the patient at the start of the treatment, preferably in immediate proximity to a vascular access which is positioned for a dialysis treatment, for example. A hose system for connection to the vascular access is then placed on the top gusseted pocket and/or the cover while the connectors are being connected. Any liquid escaping in this step cannot penetrate through the liquid barrier. The sheet may also be designed so that the moisture is absorbed by the woven fabric of the textile. The liquid sensor 2 is situated beneath the cover in a gusseted pocket formed by the folding and therefore does not come in contact with the liquid, so that no false alarm is triggered.

If all the connections have been attached, the fold can be unfolded by pulling apart the coated paper so that the sensor is exposed. This is illustrated in FIG. 5. The hose system may then be placed on the coated paper in the area of the liquid sensor. Next the fold can be folded shut again. The resulting condition is illustrated in cross section in FIG. 3 and in a view from above in FIG. 4. Any leakage occurring now on the connectors or on the vascular access can be detected by the liquid sensor. At the same time, the hose system is protected from contamination from above by the cover.

FIG. 2 and FIG. 4 show an electrical connection 5, which is to be connected and makes it possible for the covering mechanism according to the invention to be connected to a device for monitoring the vascular access of a patient, which may be integrated into a dialysis machine, for example. The electrical connection points 5 are disposed between two kinked edges. FIG. 6 shows a covering mechanism completely folded together with a total of five kinked edges and the three gusseted pockets resulting from that after folding.

A covering mechanism having at least five kinked edges also has two gusseted pockets, which open toward the top when the coated paper is unfolded. The middle pocket opens toward the bottom in the direction of the patient. The coated paper 1 is sterile at the start of the treatment, when it is removed from the package. It is then used to cover the patient; the surface is exposed toward the surroundings and may thus become contaminated, while the hose system with the connectors is protected in the bottom gusseted pocket 10, which also contains the liquid sensor 2. If operations must be performed on the connectors during the treatment, the hose system must be removed from the gusseted pocket, and in the case of covering mechanism, such as that shown in FIG. 1, for example, placed on an area that is exposed to the hospital environment. The covering mechanism shown in FIG. 6 has a gusseted pocket without a liquid sensor 2; it may remain folded up during the treatment and thus remain protected. If manipulations must be performed on the hose system, then this gusseted pocket should be folded up and the hose system placed on the coated paper 1 that has remained protected. No false alarm is triggered if leakage occurs during manipulations.

FIG. 7 shows a view of a sheet 1 with a liquid sensor 2. The liquid sensor 2 has a plurality of conductors 6, which may be woven into the woven fabric of the liquid sensor 2, for example. The conductive threads make contact at the points of intersection marked with circles, thereby forming two conductor loops. Die electrical contacts 5 are designed so that they can be connected to a device for monitoring the vascular access.

FIG. 8 shows a view of a sheet 1 with a liquid sensor 2 in the unfolded state. A barrier 7 is set up at the side of the liquid-sensitive area. It consists of an absorbent material without a measurement function. Liquid sensor 2 and barrier 7 are disposed without contact. In manipulations on the vascular access or the connectors during or before the treatment, this barrier prevents any leaking fluid from being able to penetrate into the fold at the side and come in contact with the liquid sensor 2 and thereby trigger a false alarm.

In FIG. 9 shows a cross-section through the coated paper 1 described in conjunction with FIG. 9. The liquid sensor 2 is folded in.

1. A covering mechanism for covering a hose system that can be connected to a patient (3), comprising a sheet (1) with a liquid sensor (2) and a first kinked edge (4), wherein the liquid sensor (2) and the first kinked edge are disposed on the sheet (1) in such a way that the liquid sensor (2) is disposed in a gusseted pocket formed by the fold when the sheet (1) is folded along the kinked edge.

2. The covering mechanism according to claim 1, characterized in that the sheet (1) has a second kinked edge (4′), which runs mostly parallel to the first kinked edge (4) and is designed in the opposite direction from the first kinked edge (4) such that a zigzag fold with two gusseted pockets is formed by folding of the sheet (1) along the first and the second kinked edges (4, 4′).

3. The covering mechanism according to claim 1, characterized in that the sheet (1) has at least four parallel kinked edges (4, 4′), wherein neighboring kinked edges (4, 4′) are always formed in opposite directions and one zigzag fold and at least three gusseted pockets are formed by the folding.

4. The covering mechanism according to claim 1, characterized in that the gusseted pocket formed by the folding is wider than the hose system (3) to be covered.

5. The covering mechanism according to claim 1, characterized in that the liquid sensor (2) sits at most on one kinked edge (4, 4′) of the sheet (1).

6. The covering mechanism according to claim 1, characterized in that the liquid sensor (2) and the sheet (1) are joined to one another.

7. The covering mechanism according to claim 1, characterized in that the liquid sensor (2) is integrated into the sheet (1).

8. The covering mechanism according to claim 1, characterized in that the liquid sensor (2) has an electrically conductive structure (6).

9. The covering mechanism according to claim 8, characterized in that the electrically conductive structure (6) is woven into or printed on the liquid sensor (2).

10. The covering mechanism according to claim 8, characterized in that the electrically conductive structure (6) has electrical contacts (5) for connecting to a device for monitoring an access to a patient.

11. The covering mechanism according to claim 1, characterized in that the bottom side of the sheet is impermeable to liquid.

12. The covering mechanism according to claim 1, characterized in that the sheet (1) comprises a textile, an absorbent film and/or a nonwoven.

13. The covering mechanism according to claim 1, characterized in that at least one side of the sheet (1) is sterile.

14. The covering mechanism according to claim 1, characterized in that a barrier (7) made of an absorbent material without a measurement function is applied to the sheet (1) at the edge of the liquid sensor (2) to prevent liquid from penetrating into the gusseted pocket at the side.

15. A device for monitoring an access to a patient for a system with which a fluid is supplied to a patient and/or a fluid is withdrawn from a patient through a hose system (3), in particular by monitoring the connectors of the hose system in an extracorporeal blood treatment, characterized in that the monitoring device is connected to a covering mechanism according to claim 1.
